(19) United States
(12) Patent Application Publication
(10) Pub. No.: US 2014/0329277 A1
(43) Pub. Date: Nov. 6, 2014

(54) METHOD FOR CULTURING CELLS IN ORDER TO PRODUCE SUBSTANCES

(71) Applicant: Hoffmann-La Roche Inc., Nutley, NJ (US)

(72) Inventors: Thomas Link, Penzberg (DE); Ruth Essers, Aachen (DE); Kerstin Zorner, Kerpen (DE); Jochen Gatgens, Julich (DE); Thomas Noll, Julich (DE); Christian Wandrey, Julich (DE)

(73) Assignee: HOFFMANN-LA ROCHE INC., Nutley, NJ (US)

(21) Appl. No.: 14/160,336

(22) Filed: Jan. 21, 2014

Related U.S. Application Data
(63) Continuation of application No. 10/535,581, filed on Jan. 17, 2006, now abandoned.

(30) Foreign Application Priority Data
Nov. 27, 2002 (DE) 10255508.7

(51) Int. Cl. C07K 14/705

(52) U.S. Cl. CPC C07K 14/70596
USPC 435/69.6

(57) ABSTRACT

The invention concerns a method for culturing cells in order to produce substances. According to the invention a cell line producing substances is cultured while feeding a nutrient medium in such a manner that glucose limitation occurs in the culture solution. The degree of glucose limitation DGL=qGlc/qGlcₘₐₓ (qGlc=observed current specific glucose consumption rate; qGlcₘₐₓ=maximum known specific glucose consumption rate for these cells). DGL is between the limits 0 and 1, where 0 means complete limitation and 1 means no limitations or complete glucose excess. According to the invention DGL is larger or equal to the DGL which only leads to the maintenance of the cell and ≦0.5.

The invention concerns a method for culturing cells in order to produce substances according to the precharacterizing portion of claim 1.

Cell cultures are used in fermentative processes to produce substances and in particular proteins. A distinction is made between processes in which the cell cultures are genetically unmodified and form their own metabolic products and processes in which the organisms are genetically modified in such a manner that they either produce a larger amount of their own substances such as proteins or produce foreign substances. The organisms producing the substances are supplied with a nutrient medium in this process which guarantees the survival of the organisms and enables the production of the desired target compound. Numerous culture media are known for these purposes which enable a fermentation. One of the most important components of the culture media is glucose. According to the prior art one regularly endeavours to maintain a minimum concentration of glucose in a fermentation preparation in order to optimize the yield of the target compound. The Japanese Patent Application 001 101 882 A discloses a culturing process for mammalian cells in which a minimum concentration of 0.2 mmol/l glucose is maintained. U.S. Pat. No. 5,443,968 discloses a culturing process in which a glucose limitation takes place. However, the process does not result in a higher specific production rate of the cells compared to non-limitation feeding.

The object of the invention is to create a process for culturing cells which increases the productivity of an individual cell with regard to the product and enables high cell densities. It should enable a high space/time yield of product.

The process should be particularly simple to carry out, be associated with a minimum effort for measuring and control and be particularly economic.

On the basis of the precharacterizing portion of claim 1, the object is surprisingly achieved by culturing a cell line producing substances while feeding a nutrient medium in such a manner that glucose limitation occurs in the culture solution. The degree of glucose limitation can be defined as the ratio of the observed specific glucose consumption rate to the maximum known specific glucose consumption rate for these cells. The degree of glucose limitation DGL=qGlc/qGlcₘₐₓ (qGlc=currently observed specific glucose consumption rate; qGlcₘₐₓ=maximum known specific glucose consumption rate for these cells). DGL lies within the limits between DGL_{maintenance} and 1 where DGL_{maintenance} denotes complete growth limitation and 1 denotes no limitation whatsoever or complete glucose excess.

Glucose limitation is associated with a continuous decline in the residual glucose concentration to a stationary concentration in the culture solution which is more than 0 mmol/l, but less than 1 mmol/l and preferably less than 0.5 mmol/l. It is observed that lowering the DGL can result in a further increase in the live cell density in the culture vessel. As the glucose limitation increases the cell density then converges towards a maximum value. As a result the degree of glucose limitation converges to a minimum value; the DGL according to the invention being larger than or equal to the DGL which leads to the maintenance of the cell (maintenance metabolism) DGL_{maintenance}=qGlc_{maintenance}/qGlcₘₐₓ (qGlc_{maintenance}=observed specific glucose consumption rate in the case of pure maintenance metabolism; qGlcₘₐₓ=maximum known specific glucose consumption rate for these cells) and is less than 0.5, preferably less than 0.4 and particularly preferably less than 0.3.

However, it is characteristic that the cell concentration in the solution does not decrease when the glucose concentration decreases. As the glucose limitation increases i.e. the DGL value decreases, the specific productivity of a cell increases. Since the live cell density in the culture vessel does not decrease, this leads to an increase in the space/time yield. The occurrence of glucose limitation is phenomenologically associated with a reduction in the rate of specific lactate formation. The lactate formation rate converges to a minimum value. As a result the residual lactate concentration in the culture vessel decreases to zero as a maximum. Hence glucose limitation is associated with a conversion of the cell metabolism.

In this connection it is important that there is no other limitation by other substrates before the onset of glucose limitation. Hence the growth medium must be such that glucose is limited first.

The method according to the invention increases the space/time yield at a given cell density. The method according to the invention reduces the amount of glucose that is available per cell in such a manner that glucose is mainly used in maintenance metabolism and thus for the product and less for cell growth. In this connection the method according to the invention does not require a regulation of glucose feeding and hence the method is particularly simple since a laborious glucose regulation can be omitted. Since less inflow of medium is necessary, costs for glucose are saved because less glucose is required. Moreover, a very high product concentration is achieved. This can lower the processing costs. In particular the method according to the invention enables an increase in the production of proteins without having to additionally genetically modify a cell line in order to implement the method according to the invention. The increase in the product titre enables the production of a desired amount of products in a smaller culture volume which results in lower capital expenditure.

The method according to the invention can be carried out using the following process steps:

The cells should be preferably cultured in a continuous process with cell retention e.g. spin filters (perfusion culture). All standard types of culture vessels such as stirred tanks, and cell retention mechanisms such as spin filters, ultrasound or settlers are suitable for this. The culture system should preferably enable high cell densities. Cell retention is preferable so that the cell density cannot decrease when glucose limitation occurs. As a result the DGL is further reduced as the live cell density increases and the glucose feeding remains constant. The high cell density enables the DGL to be reduced below a value of 0.4 at a set flow rate of the order of magnitude of the maximum growth rate. Thus for example flow rates of 0.03-0.05 h⁻¹ can be used for the CHO MUC2-GFP-C-term cell as well as for the CHO/MUC1-IgG2a PH3744/25 cell.

In order to reduce the DGL the feeding strategy with glucose can consequently be as follows: The amount of fed glucose is not increased as the live cell density increases in order to avoid glucose limitation. Rather the amount of fed glucose is kept constant during the process from the start. The amount of fed glucose should be selected such that the DGL falls below the required values i.e. a DGL of less than ≦0.5, preferably ≦0.4 and particularly preferably ≦0.3. As a result the amount of fed glucose is preferably not more than 50%, particularly preferably not more than 35% of that which the expected live cell count can maximally consume in the system in the case of a conventional non-glucose-limiting process control. After conversion of the cell metabolism (lactate metabolism and productivity) the amount of fed glucose can be slowly increased but should not enable a DGL of more than 0.5 and preferably more than 0.4. This results in a further increase in the live cell density with a constant high productivity and thus an increased space/time yield. In a continuous process the amount of fed glucose can be influenced by the media inflow rate and the glucose concentration in the feeding medium. It is important that the mass flow of fed glucose during the process is not increased or only to such an extent that the DGL reaches or falls below a value of less than 0.5, preferably less than 0.4 and this value is then no longer exceeded.

Advantageous further developments of the invention are set forth in the dependent claims.

Details of the invention are illustrated in the following.

The figures show examples of experimental results.

Figure legends:

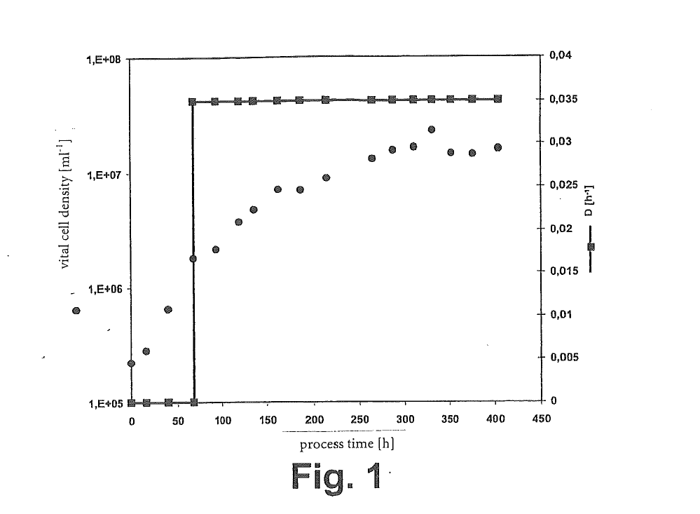

FIG. 1: Increase in the vital cell count [ml⁻¹] and plot of the media flow rate [h⁻¹] against the process time [h] for the production of MUC1-IgG2a from CHO MUC1/IgG2a PH3744/25 cells in a perfusion reactor.

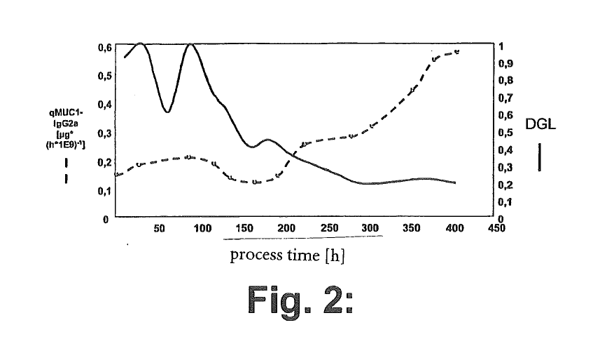

FIG. 2: Specific productivity of MUC1-IgG2a [μg/h*E9 cells] and DGL versus the process time in a perfusion reactor.

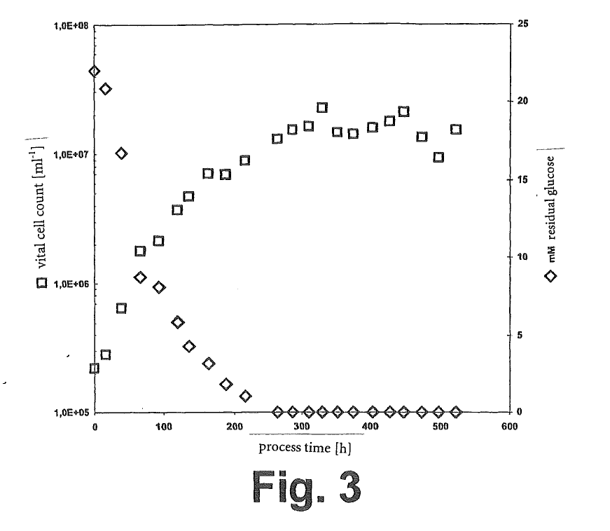

FIG. 3: Increase in the vital cell count [ml⁻¹] and mM residual glucose plotted against the process time [h] for the production of MUC1-IgG2a from CHO MUC1/IgG2a PH3744/25 cells in a perfusion reactor.

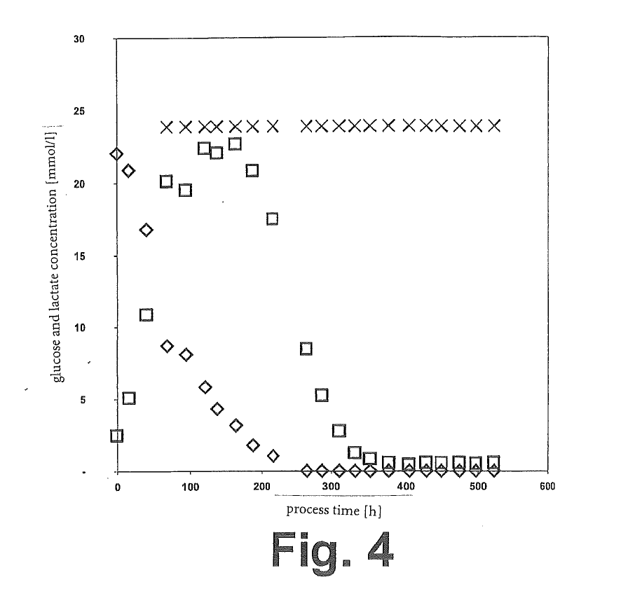

FIG. 4: Glucose and lactate concentration as well as the concentration of glucose in the media inflow [mmol/l] plotted against the process time [h] for the production of MUC1-IgG2a from CHO MUC1/IgG2a PH3744/25 cells in a perfusion reactor.

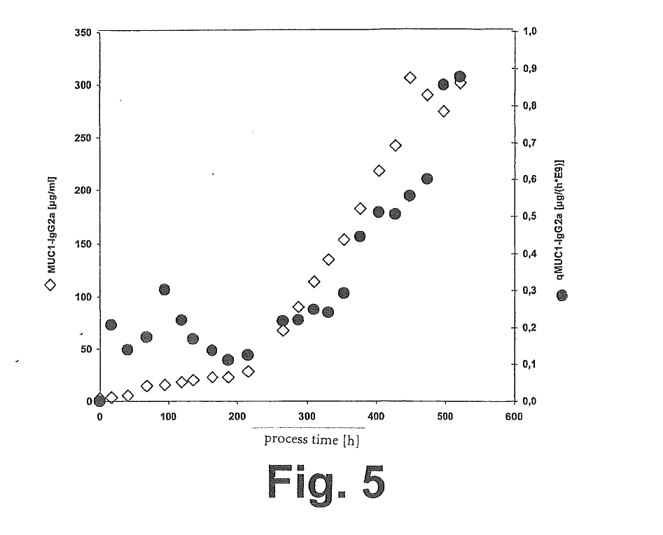

FIG. 5: Increase in the concentration of MUC1-IgG2a [μg/ml] and qMUC1-IgG2a [μg/h*E9 cells] versus time [h] for the production of MUC1-IgG2a from CHO MUC1/IgG2a PH3744/25 cells in a perfusion reactor.

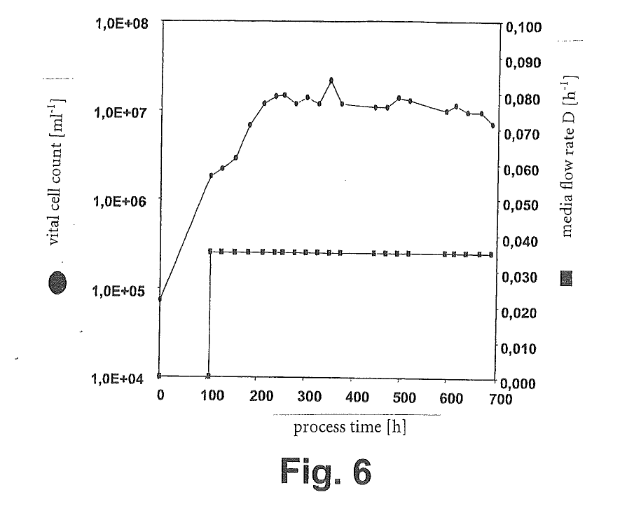

FIG. 6: Increase in the vital cell count [ml⁻¹] and plot of the media flow rate [h⁻¹] versus the process time [h] for the production of MUC2-GFP-C-term from CHO MUC2-GFP-C-term cells in a perfusion reactor.

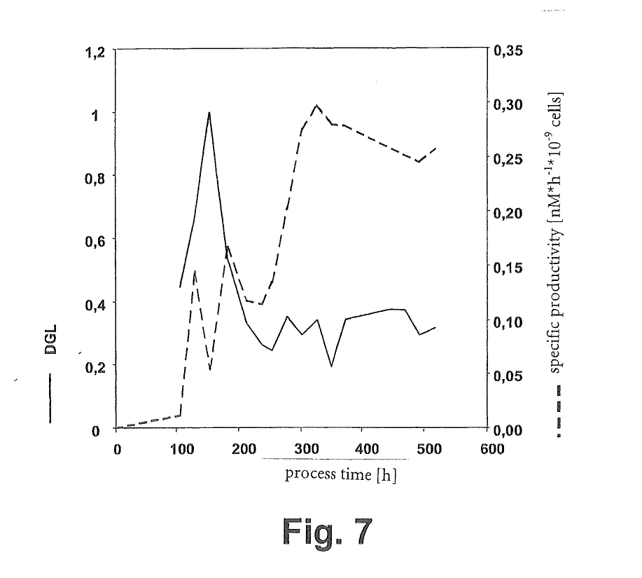

FIG. 7: Specific productivity of MUC2-GFP-C-term [nmol/(h*E9 cells)] and DGL versus the process time in a perfusion reactor.

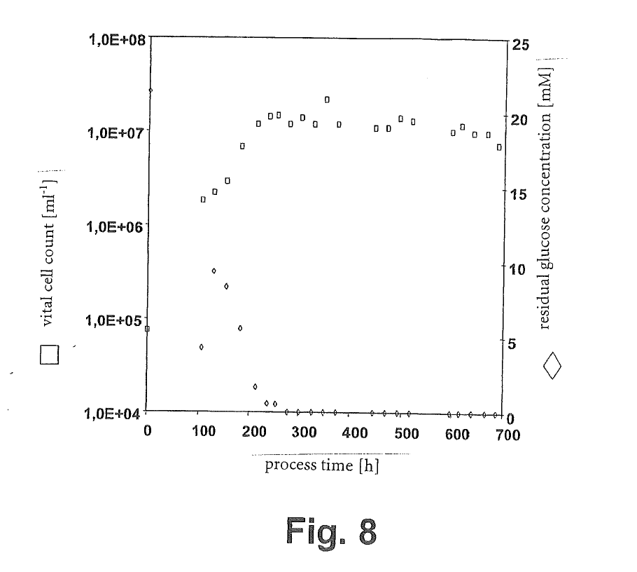

FIG. 8: Increase in the vital cell count [ml⁻¹] and residual glucose [mM] plotted against the process time [h] for the production of MUC2-GFP-C-term from CHO MUC2-GFP-C-term cells in a perfusion reactor.

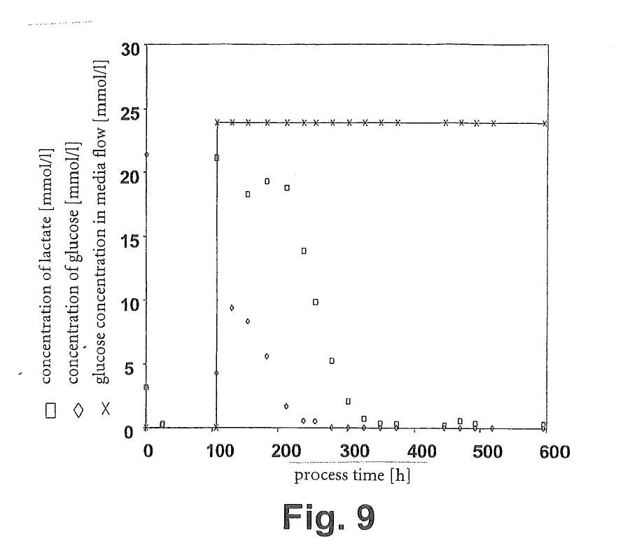

FIG. 9: Glucose and lactate concentration as well as the concentration of glucose in the media inflow [mmol/l] plotted against the process time [h] for the production of MUC2-GFP-C-term from CHO MUC2-GFP-C-term cells in a perfusion reactor.

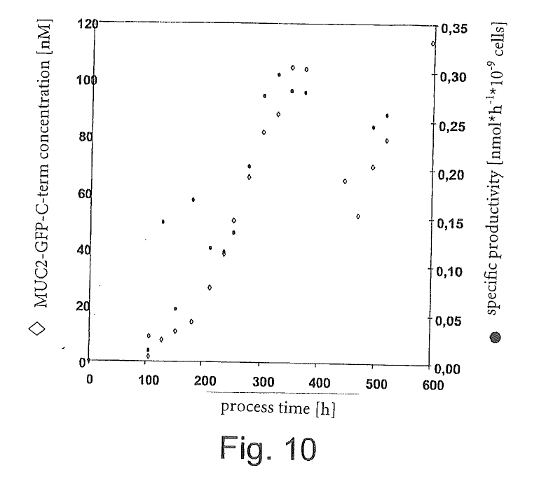

FIG. 10: Increase in the concentration of MUC2-GFP-C-term [nM] and qMUC2-GFP-C-term [nmol/(h*E9 cells)] versus time [h] for the production of MUC2-GFP-C-term from CHO MUC2-GFP-C-term cells in a perfusion reactor.

In addition table 1 shows the experimental data obtained from the use of the method according to the invention with the CHO MUC1/IgG2a PH 3744 cell.

Table 2 shows the experimental data obtained from the use of the method according to the invention with the CHO MUC2-GFP-C-term cell.

The procedure according to the invention can be carried out with various production cell lines. The cell lines can be used as a wild-type or as genetically modified recombinant cells. The genetic modification can for example take place by inserting additional genes of the same organism or of another organism into the DNA, or a vector or it can be the amplification of the activity or expression of a gene by incorporating a more effective promoter for example from CMV. The genes can code for various proteins, for example for proteins such as fusion proteins or antibodies.

The following cell lines are mentioned as examples:

Mammalian cells such as CHO cell lines such as CHO-K1, BHK such as BHK-21, hybridoma, NS/0, other myeloma cells and insect cells or other higher cells. The use of cells whose production is preferably not coupled to growth is particularly preferred.

A recombinant CHO cell line whose productivity can be increased by the procedure according to the invention is the cell line CHO MUC1/IgG2a, PH 3744/25 which can be used to secrete the glycoprotein MUC1-IgG2a. Another CHO cell line i.e. CHO MUC2-GFP-C-term is capable of secreting an increased amount of a fusion protein MUC2-GFP-C-term when it is subjected to the procedure according to the invention.

In principle any glucose-containing medium can be used as the culture medium which is not limiting with regard to other components. ProCHO4-CDM is mentioned as an example. Media based on known formulations such as IMDM, DMEM or Ham's F12 can also be used which have been optimized for the procedure according to the invention in such a manner that only glucose limitation occurs. This can for example be achieved by having a higher concentration of the other components relative to glucose. In general it is also possible to add the glucose separate from the medium.

The pH is preferably between 6.7-7.7, particularly preferably between 7-7.3. However, other pH ranges are also conceivable.

The temperature range is preferably between 35° C.-38.5° C., particularly preferably at 37° C. for CHO MUC1-IgG2a. Other temperature ranges are also conceivable such as <35° C. at which the product is not irreversibly destroyed.

Substances such as glycoproteins, fusion proteins, antibodies and proteins in general can be produced using the culturing methods according to the invention of which for example MUC1-IgG2a, MUC2-GFP-C-term, EPO, interferons, cytokines, growth factors, hormones, PA, immunoglobulins or fragments of immunoglobulins can be mentioned.

FIG. 1 shows the time course of the live cell density (cv) of CHO/MUC1-IgG2a cells and the media flow rate (D) versus the process time (h) in a perfusion reactor. In this figure:
▪ is the media flow rate (1/h) and
the live cell density (1/ml).

FIG. 2 shows the specific productivity of MUC1-IgG2a (qMUC1-IgG2a) and DGL versus the process time in a perfusion reactor.
----- is the specific productivity (μg/hE9 cells),
— DGL (degree of glucose limitation).

FIG. 3 shows a graph in which the vital cell count [ml⁻¹] is plotted on the left side and the concentration of residual glucose [mM] is plotted on the right side against the process time [h] for the production of MUC1-IgG2 in CHO MUC/IgG2a PH3744/25.
□ is the vital cell count and
⋄ glucose.

In FIG. 4 the glucose and lactate concentration as well as the glucose concentration in the media inflow [mmol/l] are plotted against the process time [h]. In this figure the curves with
□ are lactate concentration curves and
⋄ are glucose concentration curves

x 23.9 mmol/l concentration of glucose in the media inflow (flow rate of D=0.035 h⁻¹).

In FIG. 5 the concentration of MUC1-IgG2a [μg/ml] is plotted on the left side and qMUC1-IgG2a [μg/(h*E9 cells)] is plotted on the right side of the graph against time [h]. In this figure
is the specific productivity q of MUC1-IgG2a (μg/hE9 cells) and
⋄ is the concentration of MUC1-IgG2a (mg/l).

FIG. 6 shows the time course of the live cell density (cv) of CHO/MUC2-GFP cells and the media flow rate (D) versus process time (h) in a perfusion reactor. In this figure
▪ is the media flow rate (1/h) and
is the live cell density (1/ml).

FIG. 7 shows the specific productivity of MUC2-GFP-C-term (qMUC2-GFP-C-term) and DGL versus the process time in a perfusion reactor. In this figure
----- is the specific productivity (nmol/hE9 cells),
— is DGL (degree of glucose limitation).

FIG. 8 shows a graph in which the vital cell count [ml⁻¹] is plotted on the left side and the concentration of residual glucose [mM] is plotted on the right side against the process time [h] for the production of MUC2-GFP-C-term in CHO MUC/IgG2a PH3744/25. In the graph
□ is the vital cell count and
⋄ is glucose.

In FIG. 9 the glucose and lactate concentration as well as the glucose concentration in the media inflow [mmol/l] are plotted against the process time [h]. In this figure the curves with
□ are lactate concentration curves and
⋄ are glucose concentration curves

x 23.9 mmol/l concentration of glucose in the media inflow (flow rate of D=0.035 h⁻¹).

In FIG. 10 the concentration of MUC2-GFP-C-term [nM] is plotted on the left side and qMUC2-GFP-C-term [nmol/(h*E9 cells)] is plotted on the right side of the graph against time [h]. In this figure
is the specific productivity q of MUC2-GFP-C-term (nmol/hE9 cells) and
⋄ is the concentration of MUC2-GFP-C-term (nM).

FIG. 1 shows the procedure according to the invention with regard to glucose feeding as an example. A constant amount of glucose is fed into a continuous perfusion culture. In the example shown this is achieved by a constant media flow rate where the glucose concentration is constant in the media inflow. The media flow rate is not increased with increasing live cell density. The process was started as a batch before the continuous process began.

FIG. 2 shows that in this procedure the DGL decreases in the course of the process and finally reaches a value below 0.4. As this occurs the specific productivity increases and finally reaches a value which is 4-fold higher than the value before falling below the DGL value of 0.4.

FIG. 3 shows that the live cell density tends towards a maximum value which can then be maintained while the residual glucose concentration tends towards zero in the course of time. This occurs even though glucose is fed. During the lowering of the residual glucose concentration, the specific glucose uptake rate of the organisms starts to decrease. As this occurs the live cell count can still increase. In parallel with the decline in the specific glucose uptake rate, the specific lactate formation rate also decreases which initially results in a slower increase and then to a decrease in the lactate concentration in the culture vessel. Finally the lactate concentration in the culture vessel tends towards zero as shown in FIG. 4. Hence there is a considerable changeover in the cell metabolism. As shown in FIG. 5 the changeover in cell metabolism is associated with an increase in the specific productivity to about 4-fold compared to the time before the changeover in cell metabolism. The increase in the specific productivity with an at least constant or still increasing cell density during the described phase finally leads to a significant increase in the product titre in the culture supernatant as shown in FIG. 5 and thus to an increased space/time yield.

Table 1 shows data on the fermentation of MUC1-IgG2a.

Similarly to FIGS. 1 to 5, FIGS. 6 to 10 describe the results using the method according to the invention with CHO MUC2-GFP-C-term cells.

Table 2 shows data on the fermentation of MUC2-GFP-C-term.

With regard to production engineering the method according to the invention can also be operated as a fed batch (feeding process) in addition to the perfusion method described above.

In a fed-batch operation the production culture is supplied once or repeatedly or batchwise or continuously with a glucose-containing medium or a separate glucose solution in such a manner that the DGL preferably decreases below a value of 0.5, particularly preferably 0.4 and better still 0.3. A repetitive fed-batch is also possible in this case.

The process can be started in all generally known procedures in the perfusive process as well as in the fed-batch process. Thus before starting the procedure according to the invention the culture can be operated as a batch, fed-batch or continuous procedure with or also without cell retention.

TABLE 1
Data for the fermentation of MUC1-IgG2a

| process | | | glucose | | | MUC1- | qMUC1- | |
|---|---|---|---|---|---|---|---|---|
| time | cv | D | feed | glucose | lactate | IgG2a | IgG2a | |
| H | 1/ml | 1/h | mmol/l | mmol/l | mmol/l | μg/ml | μg/(h*E9) | DGL |
| 0 | 2.23E+05 | 0 | 0 | 22.07 | 2.5 | 2.62 | | |
| 16.63 | 2.83E+05 | 0 | 0 | 20.89 | 5.1 | 3.59 | 0.21 | 0.92 |
| 40.52 | 6.48E+05 | 0 | 0 | 16.75 | 10.84 | 5.77 | 0.14 | 0.99 |
| 68 | 1.78E+06 | 0 | 0 | 8.74 | 20.1 | 14.21 | 0.17 | 0.61 |
| 94 | 2.14E+06 | 0.035 | 23.89 | 8.08 | 19.48 | 15.49 | 0.30 | 1.00 |
| 120 | 3.70E+06 | 0.035 | 23.89 | 5.84 | 22.35 | 18.02 | 0.22 | 0.72 |
| 136.5 | 4.68E+06 | 0.035 | 23.89 | 4.30 | 22.02 | 19.95 | 0.17 | 0.62 |
| 163.5 | 7.02E+06 | 0.035 | 23.89 | 3.17 | 22.66 | 22.67 | 0.14 | 0.40 |
| 187.5 | 6.96E+06 | 0.035 | 23.89 | 1.79 | 20.77 | 22.44 | 0.11 | 0.44 |
| 215.5 | 8.85E+06 | 0.035 | 23.89 | 1.04 | 17.46 | 28.24 | 0.13 | 0.35 |
| 264.75 | 1.30E+07 | 0.035 | 23.89 | — | 8.45 | 67.03 | 0.22 | 0.24 |
| 287 | 1.54E+07 | 0.035 | 23.89 | — | 5.25 | 89.42 | 0.22 | 0.20 |
| 310 | 1.64E+07 | 0.035 | 23.89 | — | 2.77 | 113.28 | 0.25 | 0.19 |
| 331 | 2.27E+07 | 0.035 | 23.89 | — | 1.24 | 133.80 | 0.24 | 0.14 |
| 352.4 | 1.45E+07 | 0.035 | 23.89 | — | 0.82 | 152.87 | 0.29 | 0.21 |
| 376.3 | 1.42E+07 | 0.035 | 23.89 | — | 0.53 | 182.52 | 0.45 | 0.22 |
| 404.4 | 1.58E+07 | 0.035 | 23.89 | — | 0.44 | 218.51 | 0.51 | 0.20 |
| 428 | 1.78E+07 | 0.035 | 23.89 | — | 0.58 | 241.75 | 0.50 | 0.17 |
| 448.4 | 2.08E+07 | 0.035 | 23.89 | — | 0.55 | 305.39 | 0.55 | 0.15 |
| 473.63 | 1.35E+07 | 0.035 | 23.89 | — | 0.55 | 290.52 | 0.60 | 0.23 |
| 496.8 | 9.30E+06 | 0.035 | 23.89 | — | 0.51 | 274.94 | 0.85 | 0.33 |
| 521.82 | 1.53E+07 | 0.035 | 23.89 | — | 0.56 | 301.12 | 0.87 | 0.20 |

TABLE 2
Data for the fermentation of MUC2-GFP-C-term

| | | | | | | MUC2- | | |
|---|---|---|---|---|---|---|---|---|
| process | vital cell | | glucose | | | GFP-C- | | |
| time | count | D | feed | glucose | lactate | term | qProduct | |
| h | 1/ml | 1/h | mmol/l | mmol/l | mmol/l | nM | nmol/(h*E9) | DGL |
| 0.5 | 7.50E+04 | 0 | 0 | 21.37 | 3.12 | 0.00 | | |
| 106 | 1.80E+06 | 0 | 0 | 4.25 | 21.1 | 1.66 | 0.01 | 0.44 |
| 106.01 | | 0.035 | 23.89 | | | 8.92 | | |
| 130 | 2.20E+06 | 0.035 | 23.89 | 9.36 | | 7.71 | 0.14 | 0.66 |
| 154 | 2.90E+06 | 0.035 | 23.89 | 8.32 | 18.23 | 10.72 | 0.05 | 1.00 |
| 182.38 | 6.83E+07 | 0.035 | 23.89 | 5.58 | 19.28 | 14.08 | 0.17 | 0.53 |
| 212.9 | 1.19E+07 | 0.035 | 23.89 | 1.65 | 18.78 | 26.15 | 0.12 | 0.33 |
| 237.2 | 1.44E+07 | 0.035 | 23.89 | 0.54 | 13.84 | 38.37 | 0.11 | 0.26 |
| 254 | 1.48E+07 | 0.035 | 23.89 | 0.52 | 9.81 | 50.08 | 0.13 | 0.24 |
| 278 | 1.20E+07 | 0.035 | 23.89 | — | 5.19 | 65.63 | 0.20 | 0.35 |
| 302 | 1.40E+07 | 0.035 | 23.89 | — | 2.05 | 81.53 | 0.27 | 0.29 |
| 326 | 1.20E+07 | 0.035 | 23.89 | — | 0.7 | 88.03 | 0.30 | 0.34 |
| 349.9 | 2.16E+07 | 0.035 | 23.89 | — | 0.33 | 104.60 | 0.28 | 0.19 |
| 374 | 1.20E+07 | 0.035 | 23.89 | — | 0.26 | 104.03 | 0.28 | 0.34 |
| | | 0.035 | 23.89 | — | | 84.47 | | |
| | | 0.035 | 23.89 | — | | 75.16 | | |
| 446 | 1.10E+07 | 0.035 | 23.89 | — | 0.19 | 64.81 | | 0.37 |
| 470 | 1.10E+07 | 0.035 | 23.89 | — | 0.53 | 52.36 | | 0.37 |
| 494 | 1.40E+07 | 0.035 | 23.89 | — | 0.32 | 69.63 | 0.24 | 0.29 |
| 518 | 1.30E+07 | 0.035 | 23.89 | — | | 79.34 | 0.26 | 0.32 |
| | | 0.035 | 23.89 | — | | 93.94 | | |
| | | 0.035 | 23.89 | — | 0.35 | 104.57 | | |
| 595.8 | 1.01E+07 | 0.035 | 23.89 | — | 0.25 | 113.89 | | |

1-15. (canceled)

16. A method for producing an antibody comprising culturing recombinant Chinese Hamster Ovary (CHO) cells that produce said antibody in a glucose-containing medium under glucose limiting conditions, wherein the degree of glucose limitation (DGL) is ≦0.5.

17. The method of claim 16, wherein the DGL is ≦0.4.

18. The method of claim 16, wherein the DGL is ≦0.3.

19. The method of claim 16, wherein the CHO cells are cultured in a continuous process.

20. The method of claim 19, wherein the CHO cells are cultured in a continuous perfusion culture.

21. The method of claim 16, wherein the CHO cells are cultured in a fed-batch process.

22. The method of claim 16, wherein the feed rate of glucose is kept constant during the entire culture process.

23. The method of claim 22, wherein the feed rate of glucose is 0.03 to 0.05 h⁻¹.

24. The method of claim 16, wherein the amount of glucose fed is not more than 50% of that which the expected live cell count can maximally consume under non-glucose limiting conditions.

25. The method of claim 24, wherein the amount of glucose fed is not more than 35% of that which the expected live cell count can maximally consume under non-glucose limiting conditions.

26. The method of claim 16, wherein a glucose-containing medium is used which is not limiting with regard to other nutrient components before glucose limitation occurs.

27. The method of claim 16, wherein the cells are cultured at a pH of 6.7 to 7.7.

28. The method of claim 16, wherein glucose is added separately from other components of the culture medium.
